(19) United States
(12) Patent Application Publication
(10) Pub. No.: US 2021/0027407 A1
(43) Pub. Date: Jan. 28, 2021

(54) METHOD AND APPARATUS FOR THE UNIFIED EVALUATION, PRESENTATION AND MODIFICATION OF HEALTHCARE REGIMENS

(71) Applicants: Erick Von Schweber, Foster City, CA (US); Linda Von Schweber, Foster City, CA (US)

(72) Inventors: Erick Von Schweber, Foster City, CA (US); Linda Von Schweber, Foster City, CA (US)

(21) Appl. No.: 17/067,452

(22) Filed: Oct. 9, 2020

Related U.S. Application Data
(63) Continuation of application No. 16/507,990, filed on Jul. 10, 2019, now abandoned.
Application No. 16/507,990 is a continuation of application No. 12/395,466, filed on Feb. 27, 2009, now abandoned.

(51) Int. Cl. G06Q 50/22; G06Q 10/06; G06Q 40/08; G16H 50/30

(52) U.S. Cl. CPC G06Q 50/22; G06Q 10/06; G16H 10/20; G16H 50/30; G06Q 40/08

(57) ABSTRACT

A method and apparatus for modifying a healthcare regimen for a patient, the method comprising: determining an individual patient's healthcare regimen, the healthcare regimen having at least three healthcare components; storing data representing the healthcare regimen in a computing system; computing, by a processor in the computing system, a health care regimen risk level value for the health care regimen; identifying a modified healthcare regimen better suited to the individual patient based in part on the computed risk-level value; and modifying the healthcare regimen of the individual patient.

### CROSS-REFERENCE TO RELATED APPLICATIONS

This patent application is a Continuation of pending U.S. patent application Ser. No. 16/507,990, filed Jul. 10, 2019 and entitled METHOD AND APPARATUS FOR THE UNIFIED EVALUATION, PRESENTATION AND MODIFICATION OF HEALTHCARE REGIMENS, which in turn is a continuation of U.S. patent application Ser. No. 12/395,466, filed Feb. 27, 2009, also entitled METHOD AND APPARATUS FOR THE UNIFIED EVALUATION, PRESENTATION AND MODIFICATION OF HEALTHCARE REGIMENS, which in turn claims the benefit of U.S. Provisional Application Ser. No. 61/932,166, filed on Feb. 28, 2008, each of which are incorporated herein by reference in their entirety.

### FIELD

The field of the invention relates generally to healthcare and life science computer systems and pertains particularly to a method and apparatus for the unified evaluation, presentation and modification of healthcare regimens.

### BACKGROUND

A healthcare regimen is a specification or reference to one or more healthcare components utilized together or in concert. For example, a healthcare regimen may be the set of individual drugs of a multi-drug regimen with dose information and information on how each is to be taken, and the condition(s) they are to be taken for.

Healthcare components may include but are not limited to treatment and prevention components, such as prescription drugs, over-the-counter drugs, other drugs, nutraceuticals, herbal supplements, vitamins, minerals, homeopathics, enzymes, dietary recommendations, fitness and exercise routines, programs and therapies, lifestyle recommendations, medical and health procedures, medical devices and/or device usage scenarios, etc.

Healthcare components may also include but are not limited to diagnostics and diagnoses, tests, patient models, species models, disease models, practitioners of all kinds, providers, insurance plans, facilities, etc.

A healthcare regimen specification may indicate how the components are composed to form a regimen, e.g., drugs that are to be taken together is a simple structure while a procedure that is to be performed by a clinician meeting specified requirements using equipment of a certain kind prepared in a designated fashion at a facility of a certain sort is a more complex example. Note that a structure that composes healthcare components may itself be complex, e.g., a nested tree or graph with intricate relations between the components, a nested structure of structures or regimen of regimens through multiple levels of composition.

A drug regimen is an example of a healthcare regimen, where the evaluation of the regimen is with respect to the aspects of primarily adverse drug effect risks. Aspects may be risks, benefits as well as neutral properties, where the classification of an aspect in this regard is determined by context. Examples of related aspects may include potential adverse effect risks, absence of adverse effects, ease of taking (e.g., a small, easy to swallow pill vs. an injection or IV), frequency of dosing, price as based on various kinds of pricing models such as retail, wholesale and actual, absolute, relative and out-of-pocket cost under a specified insurance plan, as well as whether the drug is a generic or brand name.

As many as 4,000 Americans deaths each week have been attributed to adverse drug effects, with over ten times that number experiencing a life threatening adverse effect each week. Adverse Drug Effects, or ADEs as they are abbreviated, pose a problem of crisis proportions.

Those versed in the art of healthcare information technology are aware of the risks of pair-wise drug interactions, also known as drug-drug, drug-food and drug-herb interactions; these are risks that existing drug interaction checkers within the art—computer-based tools—calculate on a pair-by-pair basis across the drugs in a single multi-drug regimen.

Less well known are the risks of side effects posed by drugs individually. Drug monographs, drug inserts and drug information sheets provide qualitative information in non-structured textual form on proper dosing, indications, contraindications, common side effects, precautions and interactions with other drugs, herbs and foods. Wolters Kluwer, a commercial entity, offers a data product called Facts & Comparisons that provides static tabular arrays where columns represent drugs belonging to the same drug class, e.g., Non-Steroidal Anti Inflammatory Drugs (NSAIDs), rows represent select symptoms that have been reported as adverse reactions to these drugs in the research literature and where a cell of the array contains a value, presented as text, representing the frequency of incidence of a side effect reaction for a given drug. For example, a row for the symptom Tachycardia intersects a column for the drug Celecoxib at a cell with the value “<2”, with the following meaning: it is found in the research literature that Tachycardia presents as an adverse reaction to Celecoxib in less than two percent of those taking it. This provides a useful reference for a prescribing physician in considering various drugs that are members of a given class to prescribe from, e.g., NSAIDs. Data providers, such as First DataBank and Wolters Kluwer, publish drug databases that incorporate individual drug side effect and drug interaction data that may be loaded into relational database management systems and applied in research, clinician, payer and consumer-facing applications.

Clinicians have been advised to perform a Drug Regimen Review (DRR) in complex and problematic cases, as are often found amongst those of advanced age who frequently are on regimens of multiple pharmaceuticals (called polypharmacy) as therapy for multiple chronic conditions. Numerous methods and multi-step processes for conducting a DRR have been discussed in the professional pharmacy literature since the early 1990s, including for example, the determination for each of a patient's medications i) the reason(s) for use; ii) the therapeutic objective; iii) the appropriateness of the medication and the appropriate dose/duration/frequency given the patient's characteristics; iv) establishment of both therapeutic and adverse consequences of the medication; and v) a cost benefit analysis.

Drug interaction checkers have been discussed in this literature as a method to support (iv). Unfortunately, the death toll, disability and suffering from adverse drug effects has continued to rise despite DRR processes and the availability of drug side effect data products and drug interaction checkers.

### SUMMARY

A method and apparatus for the unified evaluation, presentation and modification of healthcare regimens are disclosed. According to one embodiment a computer-implemented method comprises computing one or more compound factors for a healthcare regimen. The compound factors include one of compound risks and compound benefits. The one or more compound factors are provided to a client.

One or more adverse effects are provided to the client with a corresponding compound conditional probability of the one or more compound factors.

### BRIEF DESCRIPTION OF THE DRAWINGS

The accompanying drawings, which are included as part of the present specification, illustrate the presently preferred embodiment and together with the general description given above and the detailed description of the preferred embodiment given below serve to explain and teach the principles of the present invention.

FIG. 1 illustrates one embodiment for a unified regimen evaluation, presentation and modification interface utilizing a table and graph presentation theme.

FIG. 2 illustrates one embodiment for a unified regimen evaluation and presentation interface utilizing a “tag cloud” presentation theme.

FIG. 3 illustrates a unified healthcare regimen evaluation, presentation and modification process according to one embodiment.

FIG. 4 illustrates a process for the computation and management of the unified evaluation, presentation and modification of healthcare regimens utilizing a code generator, according to one embodiment.

FIG. 5 illustrates a process for computation and management of the unified evaluation, presentation and modification of healthcare regimens utilizing the derivation of an XML representation and transformation of the representation, according to one embodiment.

FIG. 6 illustrates a process for the computation and management of the unified evaluation, presentation and modification of healthcare regimens utilizing transformation of an XML representation according to one embodiment.

FIG. 7 illustrates a process for the computation and management of the unified evaluation, presentation and modification of healthcare regimens utilizing the derivation and/or retrieval of a semantic web representation and transformation of the representation, according to one embodiment.

FIG. 8 illustrates an exemplary computing system within which a set of instructions, for causing the machine to perform any one of the methodologies discussed herein, may be executed, according to one embodiment.

FIG. 9 illustrates a unified regimen evaluation, presentation and modification interface presenting multiple kinds of aspects of a healthcare regimens, according to one embodiment.

### DETAILED DESCRIPTION

A method and apparatus for the unified evaluation, presentation and modification of healthcare regimens are disclosed. According to one embodiment a computer-implemented method comprises computing one or more compound factors for a healthcare regimen. The compound factors include one of compound risks and compound benefits. The one or more compound factors are provided to a client. One or more adverse effects are provided to the client with a corresponding compound conditional probability of the one or more compound factors.

Drug safety tooling includes drug monographs, side effect tables for the drugs belonging to a drug class, drug databases and drug interaction checkers. They have failed to adequately address the growing crisis of adverse drug effects. While a root cause analysis reveals that all drugs exhibit toxicities that accompany their therapeutic properties, the effective cause of the crisis is the inability to manage these toxicities.

Prior systems provide general guidance on a regimen; they are not personalized to the individual patient and their issues, symptoms and concerns.

Prior systems largely provide qualitative guidance that must be read, understood, analyzed, evaluated and applied. They rarely are directly actionable.

Prior systems focus on individual regimen components, e.g., a drug in isolation, or on a pair of components, e.g., a pair-wise drug interaction. Prior systems ignore the full impact of components at the regimen level, e.g., the impact of the regimen as a unit.

Prior systems in performing symptom diagnosis rarely evaluate the contribution of adverse drug effects, looking instead for primary conditions. When they do consider the role and impact of adverse effects it is limited to the impact of individual drugs in isolation or in pairs, however, never the impact of the regimen as a unit.

Prior systems focus on the flagging of problems while ignoring the resolution of those problems.

Prior systems ignore trade-offs. No healthcare regimen, such as a treatment regimen or drug regimen, is perfectly desirable in all conceivable respects or aspects. Each has its trade-offs, its pros and cons relative to a decision context. One drug regimen may present a substantial risk of hives and no risk of heart palpitations; that's a trade-off. Another may present a high risk of palpitations and no or low risk of hives; that's a different trade-off. If this second regimen is medically indicated for the same conditions as the first then a trade-off exists between the two regimens, another trade-off.

These issues are as applicable to dietary regimens as they are to drug regimens, regimens of procedures, regimens that combine varied components, e.g., diet, drugs, nutraceuticals, exercise, medical devices, etc. They also apply outside the scope of treatments, to healthcare regimens of professionals, e.g., physicians, pharmacists, specialists, nurses, surgeons, and therapists, researchers, and to facilities such as clinics, labs and hospitals, and apply to models of a patient, species and disease, to genetic and genomic tests and analyses, and more generally, to omics models, tests and therapies, to diagnostics and diagnoses in general and beyond to insurance plans to pay for any of the above. Healthcare regimens composing one or more components (elements, objects, entities, facets; or classes or groupings of these) may be considered according to one or more aspects (dimensions, attributes, criteria, and/or objectives) which may be represented formally as predicates. Trade-offs exist for individual regimens and amongst healthcare regimens with respect to one or a plurality of aspects of a given decision context.

Flagging of problematic pair-wise chemical interactions is the full and complete extent in prior systems of regimen evaluation, presentation and modification that considers more than a single regimen component. This is only for elements of drugs, foods, herbs and a limited set of patient aspects. Prior systems perform no analysis, evaluation and presentation of the combined aspects across all the components of a healthcare regimen. For example, the celebrity Heath Ledger at the time of his death was on a regimen of legally prescribed drugs taken in clinically recommended doses where none of the drugs in his regimen exhibited drug interactions. State of the art methods and tooling, including interaction checkers, were unable, and continue to be unable, to reveal that the toxicities of his drugs added up to create a significant combined risk—at the regimen level—of severe depression of lung function, the cause of his death.

The drug interaction checkers, drug class tables and drug databases of prior systems, deficient as just described, are the only prior biomedical informatics tools for supporting the established processes and procedures of drug regimen review and medication therapy management. They are unable to evaluate the combined risks of adverse drug effects across the drugs in a multi-drug regimen. Prior systems are similarly unable to identify where these combined risks stem from in terms of each drug's contribution to the overall regimen risk. Prior systems are unable to explore the impact on risks of modifications to the regimen, necessary toward identifying regimens better suited to the patient.

The present system provides for computation, analysis, evaluation, presentation and modification of healthcare regimens to function at the level of regimen synergies and not just the component level: to reveal aspects of a healthcare regimen or a plurality of them, including regimen-level aspects; to identify components that contribute to regimen-level aspects along with the extent of their contribution; to enable the exploration of modifications to a regimen; and to operate across components and aspects of all kinds and sorts, both risks and benefits, and to allow comparisons of regimens.

Consider an example evaluating a drug regimen with respect to the risk of adverse side effects. A drug regimen of Fenoprofen, Fosamax and Zomig is typical therapy for bursitis, osteoporosis and migraine headaches, respectively. The table below identifies the risk of drowsiness as a side effect of each of these drugs taken individually.

Each drug in a drug regimen presents its own risk of drowsiness.

| Drug | Drowsiness Risk | Drowsiness Risk (Boolean) |
|---|---|---|
| Fenoprofen oral | 0.13 | TRUE |
| Fosamax oral | 0.00 | FALSE |
| Zomig oral | 0.07 | TRUE |

Consider the risk of drowsiness for Fenoprofen as well as a Boolean truth value of this risk. In a research study, clinical trial or meta analysis, 13% of subjects presented drowsiness as a side effect of Fenoprofen. A probabilistic interpretation is that Fenoprofen presents a probability of 0.13 that the taker of this medication will present with drowsiness. The Boolean truth value simply says that there is a risk of drowsiness associated with Fenoprofen. In comparison note that the probability of drowsiness as a side effect of taking Fosamax is 0.00 and it's Boolean truth value is FALSE.

Consider now the evaluation of the risk of drowsiness as a side effect from this drug regimen as a whole, e.g., the cumulative or combined risk of drowsiness presented by this drug regimen as a unit.

Many methods may be used to assess the risk of drowsiness for the regimen as a whole. These include but are not limited to:
a logical operation applied to the individual Boolean risks of the components, e.g., the regimen presents a risk if any of its components present a risk (e.g., a logical “OR” of the component Boolean risks);
a count of individual Boolean risks, e.g., two of the three components of the regimen present a risk;
risk exposure, e.g., two thirds of the drugs in the regimen present a risk, or 66%; Combinatorial assessment via compound conditional probability of individual risk probabilities guided by the Law of Total Probability (see the detailed discussion below of an example for calculating combined risk);
weighted compound probability of individual risk probabilities;
Bayesian inference;
Dempster Shafer inference;
a scoring function applied to individual risk probabilities;
a computation that assesses an aspect's value for the regimen as a unit given the aspect's values for the individual components and the regimen structure;
risk values observed or reported for a regimen as a whole in cohort studies, trials, the research literature or in the community.

The prior example is now extended to consider the drowsiness risk posed by a regimen as a unit. The following table provides an example of a few of these regimen risk evaluative methods for the regimen as a unit. Each method provides valuable input to assessment: that there exists a risk of drowsiness (True), that 2 out of the 3 meds in the regimen pose a risk for drowsiness, that 66% of the regimen meds pose a risk of drowsiness, and that the combined risk of drowsiness is 0.19.

| | | | | Combined Risk |
|---|---|---|---|---|
| | Boolean | | | (Compound |
| | ORed | Risk | Risk | conditional |
| Regimen | Risk | Count | exposure | probability) |
| Fenoprofen + | True | 2 (of 3) | ⅔ = 66% | 0.19 |
| Fosamax + Zomig | | | | |

Consider the combined (combinatorial compound conditional probability) risk in more detail. The 0.19 (or equivalently 19%) risk of drowsiness associated with this regimen can be derived from the component risks as follows as guided by the Law of Total Probability. A partitioning of the event space is used to identify every way in which a patient taking this regimen is exposed to a risk of drowsiness, e.g., one way is that the patient suffers drowsiness from Fenoprofen but not from Fosamax and not from Zomig. Formally, this is the conditional probability that a patient taking all three of these drugs will experience drowsiness from Fenoprofen but not from Fosamax and not from Zomig. Formally this is expressed as P(drowsiness from regimen|no drowsiness from Fosamax AND no drowsiness from Zomig). For convenience the less formal but easier to read notation of P(Drowsiness from Fenoprofen but not from Fosamax or Zomig) is used below.

After identifying each possible way that drowsiness could occur the next step is to calculate the conditional probabilities to calculate the risk for each of these ways, then take the sum of the conditional probabilities to yield the overall compound probability. This is more formally described as taking the compound conditional probability of the component risks, or alternately, the compound probability for the regimen.

Example of computation of combinatorial compound conditional probability (combined risk) of Drowsiness from a regimen of Fenoprofen, Fosamax and Zomig:

Let P1=Drowsiness risk of Fenoprofen=0.13,

Let P2=Drowsiness risk of Fosamax=0.00, and

Let P3=Drowsiness risk of Zomig=0.07.

Then the compound conditional risk of Drowsiness from a regimen of Fenoprofen, Fosamax and Zomig

$$P ( Drowsiness from any or all of Fenoprofen , Fosamax or Zomig ) \\ = P ( Drowsiness from Fenoprofen but not from Fosamax or Zomig ) \\ + P ( Drowsiness from Fosamax but not from Fenoprofen or Zomig ) \\ + P ( Drowsiness from Zomig but not from Foneprofen or Fosamax ) \\ + P ( Drowsiness from Fenoprofen and Fosamax but not from Zomig ) \\ + P ( Drowsiness from Fosmax and Zomig but not from Fenoprofen ) \\ + P ( Drowsiness from Fenoprofen and Fosamax and Zomig ) \\ = P 1 * ( 1 - P 2 ) * ( 1 - P 3 ) \\ + P 2 * ( 1 - P 1 ) * ( 1 - P 3 ) \\ + P 3 * ( 1 - P 1 ) * ( 1 - P 2 ) \\ + P 1 * P 2 * ( 1 - P 3 ) \\ + P 1 * P 3 * ( 1 - P 2 ) \\ + P 2 * P 3 * ( 1 - P 1 ) \\ + P 1 * P 2 * P 3 \\ = 0.13 * 1.00 * 0.93 \\ + 0.00 * 0.87 * 0.93 \\ + 0.07 * 0.87 * 1.00 \\ + 0.13 * 0.00 * 0.93 \\ + 0.13 * 0.07 * 1.00 \\ + 0.00 * 0.07 * 0.87 \\ + 0.13 * 0.00 * 0.07 \\ = 0.12 + 0.00 + 0.06 + 0.00 + 0.01 + 0.00 + 0.00 \\ = 0.19 \\ = 1 - P ( Not drowsiness from Fenoprofen or Fosamax or Zomig ) \\ = 1 - [ ( 1 - P 1 ) * ( 1 - P 2 ) * ( 1 - P 3 ) ] \\ = 1 - [ 0.87 * 1.00 * 0.93 ] = 1 - 0.81 \\ = 0.19 .$$

According to one embodiment, the same example presented as Table 1 below showing an example of computation of combinatorial compound conditional probability (combined risk) of Drowsiness from a regimen of Fenoprofen, Fosamax and Zomig.

TABLE 1

| Conditional | | | |
|---|---|---|---|
| probability for | | | |
| each partition | Expressions using | Substitution of | |
| in the event space | declared variables | values | Subtotal |
| P(Drowsiness | P1 * (1-P2) * (1-P3) | 0.13 * 1.00 * 0.93 | 0.12 |
| from Fenoprofen | | | |
| but not from | | | |
| Fosamax or Zomig) | | | |
| P(Drowsiness | P2 * (1-P1) * (1-P3) | 0.00 * 0.87 * 0.93 | 0.00 |
| from Fosamax | | | |
| but not from | | | |
| Fenoprofen | | | |
| or Zomig) | | | |
| P(Drowsiness | P3 * (1-P1) * (1-P2) | 0.07 * 0.87 * 1.00 | 0.06 |
| from Zomig | | | |
| but not from | | | |
| Fenoprofen or | | | |
| Fosamax) | | | |
| P(Drowsiness | P1P2 * (1-P3) | 0.13 * 0.00 * 0.93 | 0.00 |
| from Fenoprofen | | | |
| and Fosamax but | | | |
| not from Zomig) | | | |
| P(Drowsiness | P1P3 * (1-P2) | 0.13 * 0.07 * 1.00 | 0.01 |
| from Fenoprofen | | | |
| and Zomig | | | |
| but not from | | | |
| Fosamax) | | | |
| P(Drowsiness | P2 * P3 * (1-P1) | 0.00 * 0.07 * 0.87 | 0.00 |
| from Fosamax | | | |
| and Zomig | | | |
| but not from | | | |
| Fenoprofen) | | | |
| P(Drowsiness | P1 * P2 * P3 | 0.13 * 0.00 * 0.07 | 0.00 |
| from Fenoprofen | | | |
| and Fosamax | | | |
| and Zomig) | | | |

Totals

| | | |
|---|---|---|
| P(Drowsiness | Sum of Expressions | 0.19 |
| from any or all | | |
| of Fenoprofen, | | |
| Fosamax or Zomig) | | |

This method of calculating combined risk may be verified by a cross-check. The expression [(1−P1)*(1−P2)*(1−P3)] calculates the probability of not presenting with Drowsiness on this regimen. Therefore, the expression 1−[(1−P1)*(1−P2)*(1−P3)] provides an alternate method of calculating the probability of presenting with Drowsiness from any or all drugs in this regimen. Using this alternate method and substituting values reveals the same final value of 0.19 obtained above.

These methods may be performed on a regimen at any level of composition, e.g., a dispensed drug that is composed of a plurality of ingredients may be considered a regimen itself with aspects of the drug evaluated by considering the aspects of its ingredients and applying the above methods.

Note that embodiments of risk computations may perform the computation in a single step or iterate through the components of a regimen incrementally computing the risk; these steps may also be parallelized. Further, an already computed risk for a given regimen may be revised to support evaluation of a modified regimen. For example, this may be done by incrementally computing the removal of a component's risk and then incrementally computing the additional risks of an additional component. In this fashion a re-evaluation need not start from scratch.

Given the various ways in which an evaluation may be computed now consider the presentation of such an evaluation. An evaluation of one or a plurality of healthcare regimens with respect to one or a plurality of aspects may be presented in print, electronically, optically or by other presentation forms. Some of these forms include but are not limited to text, tables and tabular representations, Web 2.0 “tag clouds”, graphs of any dimensionality, charts, animations, videos and verbal narration.

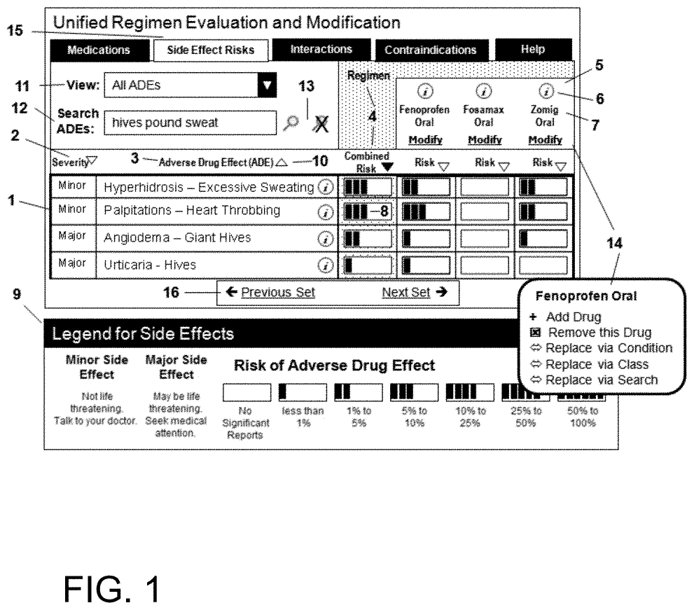

FIG. 1 illustrates one embodiment for a unified regimen evaluation, presentation and modification interface utilizing a table and graph presentation theme. The figure shows this for a single regimen with respect to a plurality of adverse drug effect aspects, where the regimen risk aspect has been derived using combinatorial, compound conditional probability (combined risk) applied to the component risks. Discussed below are the features and actions of the regimen evaluation; the computation needed to support the generation, presentation, interaction with and modification of these features and actions.

Each row, such as row 1 in FIG. 1, presents evaluation of a regimen and its components according to an aspect, such as the risk of minor Palpitations—Heart Throbbing. The column 2 reveals the severity level of the adverse effect, minor in this case. The column 3 identifies the adverse effect symptom, sign or condition, in this case formatted to first identify the effect using professional terminology, followed by a hyphen followed by terminology suitable for lay persons. The column 4 provides, in this case, statistical combined risk for the regimen as a unit; a column can just as easily provide experiential or evidence-based data for risk for the regimen as a whole obtained in laboratory testing, cohort studies or as obtained from the community using social computing methods. Column 5 provides evaluation on a regimen component (e.g., an object, element, grouping, class or category), in this case for the drug Zomig taken orally; there are similar columns for the remaining drugs in the regimen (Fenoprofen and Fosamax).

Clickable icon 6 provides access to contextual information, in this case the drug monograph for Zomig Oral. Label 7 identifies the regimen component represented by the column, in this case Zomig Oral. Histogram 8 occupies a cell of the table, in this case ascribing a 5-10% combined risk of minor palpitations to the regimen as a unit. Legend 9 provides a key by which to understand the meaning of severity levels such as Minor Side Effect and Major Side Effect, as well as the risk histograms. Clickable sort icons such as 10 are used to sort the columns of the regimen evaluation to the user's liking; by default the rows are sorted in descending order by combined risk (the “current” sort is indicated by a solid triangle; outline triangles indicate the order of sort that will be performed if the clicked).

View drop-down control 11 allows the user to control the set of rows displayed, such as All ADEs (Adverse Drug Effects), Highest Risk ADEs and Life Threatening ADEs. The Search box 12 allows the user to personalize the evaluation to those symptoms they may suspect are adverse effects; here the evaluation is searched simultaneously on “hives”, “pound” and “sweat”, thus displaying the four rows shown. Clickable icons 13 perform the search once text is entered, and respectively, clear existing search terms.

A user may enter a regimen of drugs in many ways (not shown). For example, a user may enter them directly on the evaluation from on the Medications tab, or using a context menu on the Side Effects Risks tab or via a clickable or automatic link to a persistent record of medications, local or distributed, e.g., an EHR, PHR or EMR. Users may enter their concerns or symptoms they suspect are ADEs in a variety of ways (not shown) such as entering them on a Concerns tab or via a link to a persistent record, or (as shown) by entering them directly in the Search box. The user may then i) by attending to the Combined Risk column, determine the likelihood that a concern or symptom may in fact be an ADE, such as the 5-10% risk of minor palpitations associated with this regimen. They may then ii) compare the component risks across the regimen to identify Fenoprofen as the most likely contributor to the risk of palpitations. They may then iii) use the Modify function 14 to evaluate the impact of replacing Fenoprofen with a substitute. Modifications may be iterated until the user is satisfied with the resulting changes to combined risk. For example, if the user substitutes Acetaminophen for Fenoprofen, thus lowering the combined risk for palpitations to 1-5%, but finds that the palpitations continue to clinically present following the substitution, they may reconsider the revised evaluation and observe the next most likely contributor, in this case Zomig, and explore substitutes for this component and their impact on the user's concerns.

Each component column displays a Modify context menu on mouse-over, such as context menu 14. When the user's cursor is over the Modify label a context menu appears providing the user with a plurality of actions he may take by clicking on an action. The actions shown are: the user may choose to add another drug to the regimen evaluation, he may remove a drug from the regimen evaluation or he may replace a drug in the regimen with a substitute using one of several methods (replace via Condition, via Class or via Search). For example, the user may wish to consider substitutions for Fenoprofen in order to reduce or eliminate the combined risk of palpitations (as Fenoprofen is seen to be the greatest contributor to the risk of palpitations).

Depicted in FIG. 1 is context menu 14 that is overlaid on the regimen evaluation when the user mouses over the Modify label for the drug Fenoprofen. The user may choose the action “Replace via Condition” to view all drugs that are indicated as therapy for the condition(s) for which Fenoprofen is being taken. The user may instead choose “Replace via Class” to view all drugs in the same drug class as Fenoprofen. The user may instead choose the action “Replace via Search” to perform a general search of drugs to identify a substitute. These context menu choices launch a wizard (not shown) to perform the respective action. When the wizard completes, e.g., a substitute drug is selected after viewing all drugs in the same class as Fenoprofen, the change is confirmed with the user (not shown) and the evaluation is revised and updated to reflect the drug substitution (not shown). This is similar to performing a “what-if” analysis with a spreadsheet program, e.g., making a change to a financial assumption then recalculating the spreadsheet and assessing the impact on profitability.

Other kinds of evaluations are accessible by clickable tabs, in this case tab 15 which indicates that the Side Effects Risks evaluation is currently selected. Clickable page control 16 allows the user to page through sets of rows when there are more than can be displayed on a single page.

The co-presentation of multiple regimen components, drugs in this example, with respect to one or more aspects for evaluation, provides enormous value even without consideration of the compound conditional probabilities for the regimen as a whole; seeing the individual drugs of the regimen with their aspect values enables a user to determine where the risk or risks of an adverse effect stem from, as well as the relative contributions each drug makes toward the overall regimen risk.

In another embodiment, users may expand and collapse the set of a regimen's component columns by clicking an appropriate user interface icon, thus simplifying the display When component columns are not needed and conserving screen real estate. A column for a component like a drug could even be expanded into a plurality of columns, each representing an ingredient of the drug.

In a related embodiment multiple regimens are evaluated and displayed side-by-side, optionally with the just described column collapse and expand method. This allows the user to compare combined risk across a plurality of regimens yet also enables the user to drill-down to view a regimen's components when desired.

In a related embodiment sets of regimens possessing features in common may be “grouped” or classed into sets and the expand/collapse function utilized to allow the user to compare sets of regimens, drill down into a set to see its regimens, drill down into a regimen to see its drugs and drill down into a drug to see its ingredients. Cells of the grid may then represent statistical variance across the regimens of the set, e.g., mean, median, standard deviation, minimum and maximum, all of which may be visually overlaid on the histogram or provided adjacent to it.

In a related embodiment rows may also be expanded and collapsed, for example, to “nest” the two rows related to hives in FIG. 1 under a row for “Skin Symptoms and Conditions” or drill-down into a row for ulcer to reveal multiple rows, one for each kind of ulcer at risk, e.g., a gastric ulcer.

In a related embodiment arbitrary numbers of columnar levels and row levels may be supported.

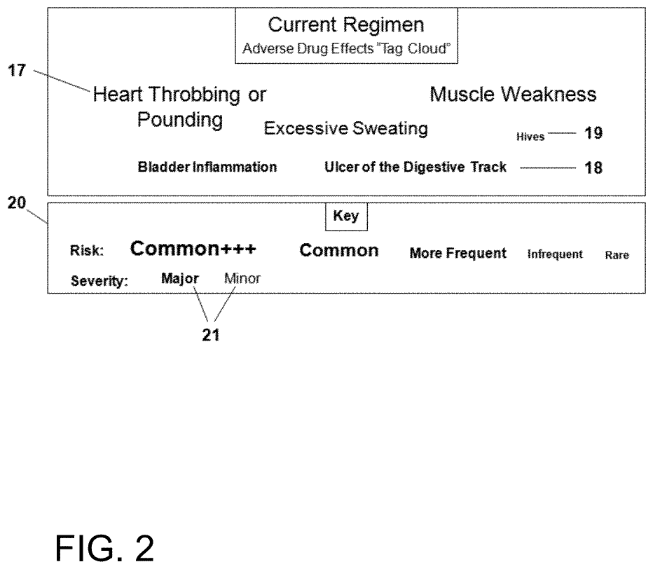

FIG. 2 illustrates one embodiment for a unified regimen evaluation and presentation interface utilizing a “tag cloud” presentation theme, where risk level is depicted proportional to font size and severity is depicted by a bold vs. non-bold font, allowing at-a-glance comprehension of the risk landscape. An adverse effect having a risk beyond common (Common+++) but of minor severity is shown in 17 as large but not bold. An effect having a less frequent risk but of major severity is shown in 18 as small and bold. An effect of rare risk and low severity is shown in 19 as small but not bold. The key to the encoding of risk and severity is shown in 20, where the two levels of bolding, representing major and minor severity, is shown in 21. A slight variation in embodiment may utilize distinct font colors to represent distinct severity levels, as for example, red for adverse effects of major severity and yellow for minor severity.

Multiple, distinct views may be interlinked, allowing the user to easily switch amongst them.

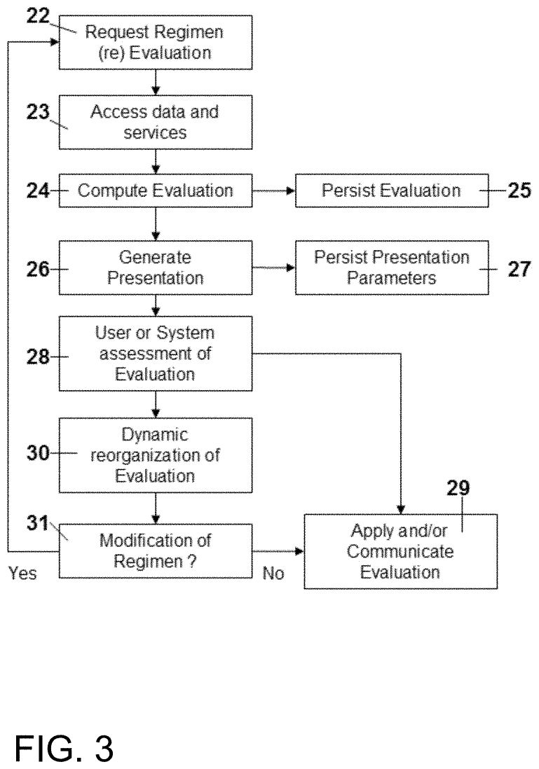

FIG. 3 illustrates a unified healthcare regimen evaluation, presentation and modification process according to one embodiment. A regimen evaluation is requested in 22; this may be initiated by a user or machine initiated by another program or process. Data and services to be employed in the evaluation are accessed in 23; these may be local or distributed. The evaluation is computed in 24. The evaluation is optionally persisted in 25. One or a plurality of presentations of the evaluation are generated in 26 for one or a plurality of users and/or other systems to assess in 28, with the optional persistence of the presentation or presentation parameters in 27. Note that the presentation may be generated appropriate to the consumer of the presentation, e.g., an XML or HTML rendering for humans or XML or a semantic web format for a machine consumer. The evaluation may be applied and/or communicated in 29. A dynamic reorganization of the evaluation may be performed in 30, for example, to view additional pages of the evaluation, to sort columns, to expand or collapse columns or rows, to change the view, etc. The reorganized evaluation may be applied and/or communicated in 29. If a modification of the evaluation is desired in 31 then a re-evaluation is requested in 22. Re-evaluations may streamline 23-27 by, for example, recalculating combined risk by removing the risk of a removed drug from the regimen's combined risk and incrementing the combined risk with the addition of an added drug.

Consider now the computational methods to realize the evaluation, presentation and modification methods and system just discussed. Computation is required to generate a healthcare regimen evaluation and its presentation and manage it and modifications to these. These computational methods operate for arbitrary and heterogeneous healthcare regimens.

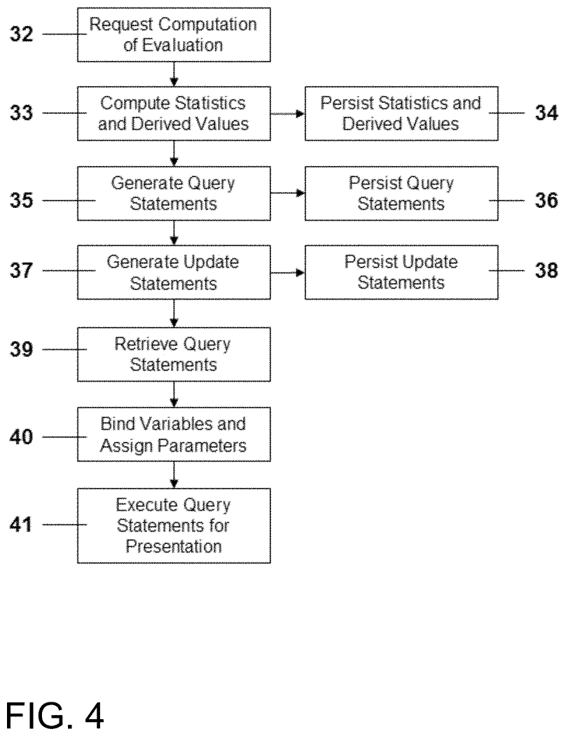

In one method, depicted in FIG. 4, a code module called a code generator is invoked upon each regimen evaluation or re-evaluation. The generator produces a set of query statements 35 that, when executed against a normalized relational database schema, derive the desired presentation schema and presentation format, including formats that may depart from the relational model, e.g., not in First Normal Form. The generator may also generate update statements 37 to reflect modifications (made to the regimen evaluation subsequent to presentation) back to the normalized relational schema.

To compute a presentation specification, (e.g., a populated data structure of the required format, that may be visualized, for example, as illustrated in FIG. 1), the code generator produces query statements that appropriately join, select and project against a relational schema of multiple relations that are in, at the very least, first normal form, but more typically Boyce-Codd normal form. One relation may manage data on a plurality of routed drugs, with one tuple for each routed drug (a routed drug is a chemical drug formulation administered using a specified route, e.g., in FIG. 1 Zomig Oral is routed drug 6, that is, the drug formulation Zomig administered orally); call this Routed_Drug_R.

Another relation may manage a plurality of medical conditions and symptoms, with one tuple for each condition/symptom; call this Condition_R. Another may manage a relationship between routed drugs and the conditions that have been reported as adverse drug effects for each of these routed drugs with one tuple per ADE per drug; call this ADE_R. A tuple of this relation could manage the assertion that, for example, minor heart palpitations is reported as a side effect of taking Fenoprofen in between five and ten percent of those taking it. Another relation may manage the drugs in a patient's drug regimen, one tuple for each drug; call this Regimen_Drug_R. Another relation may manage the side effect concerns of the patient, one tuple for each concern; call this Concern_R.

In FIG. 1 the search box 12 allows the user to enter these concerns. One of the query statements generated by the generator, when that statement is executed, may return a data structure that represents the four rows of data visualized in FIG. 1, such as row 1. The data this row represents is not directly managed in the underlying RDBMS. For example, the ADE symptom 3 in FIG. 1 may be managed by relation Condition_R; the value of combined risk that is plotted by histogram 8 may be derived by a calculation operating on a group of rows of relation ADE_R; the value of risk that is plotted as a histogram for each drug column may be managed by a unique tuple of relation ADE_R one tuple per ADE per drug. That this particular regimen evaluation requires three drug columns, such as drug column 5 in FIG. 1 is derived from relation Regimen_Drug_R.

The code generator produces a query statement specific to this regimen evaluation and its seven columns such that when the generated query statement is executed it returns a data structure populated with these seven columns and four rows. A generated query statement may include, for example, a SQL function to map a calculated combined risk value to formatting commands that, when executed, produce the corresponding histogram. Similarly, the generator may generate a query statement to provide, when executed, the required header information, e.g., a drug name for each drug column (7 in FIG. 1) or a snippet of Javascript code that supports the Modify action 14 in FIG. 1.

After the generator generates said statements the statements may be passed to the next module, or persisted (36, 38) and references to them passed back to the invoker which may then retrieve said statements 39. A late binding of variables and parameter assignments is performed 40. The bound statements are executed 41 producing the evaluation and its presentation. Computations to reflect regimen-level aspects, such as combined risk of adverse effects for the regimen as a unit, may be performed 33 and persisted 34 to the normalized relational schema prior to generator invocation or, alternatively, may be provided by the generator as part of the generated statements (as just described) which will be subsequently executed by the invoker.

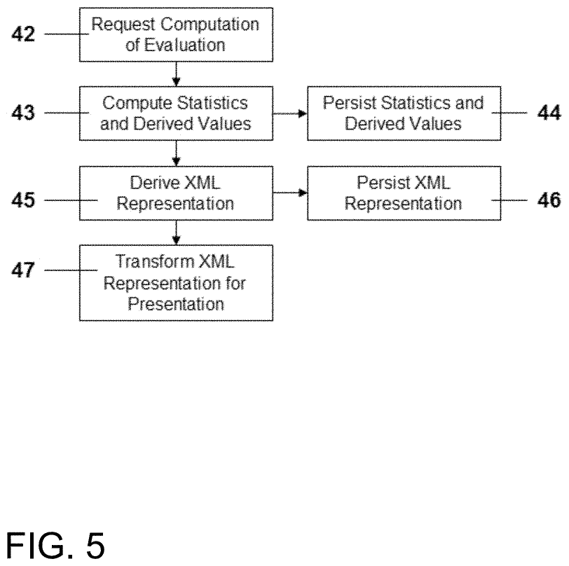

Another method, depicted in FIG. 5, employs a set of parameterized statements, expressed in SQL, SQL/XML or XQuery, to derive an XML representation 45 from the normalized relational schema when a regimen evaluation is requested. The XML representation codifies relational tuples and attributes uniformly as XML elements, allowing for regimen evaluations and presentations to be instantiated with heterogeneous numbers and types of components. A transformation module uses XSLT or XQuery statements to transform the derived XML representation 47, either passed or persisted 46 and retrieved, into the final form of the evaluation for presentation, which may be XML, XHTML, or HTML. Event handlers, developed statically and bound into the transformed XML or XHTML, process modification events to the regimen or its evaluation/presentation and update the underlying normalized relational schema.

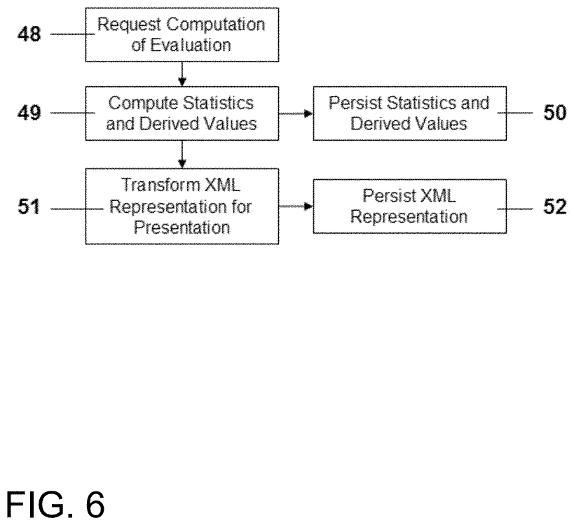

Another method, depicted in FIG. 6, manages the underlying patient and/or drug database data directly and natively as XML rather than as a normalized relational schema. XSLT or XQuery statements are invoked when a regimen evaluation is requested, said statements 51 transform the native XML into the final form of the evaluation for presentation, which may be XML, XHTML or HTML. Event handlers developed statically, process modification events to the regimen or its evaluation/presentation and update or version the underlying XML files or XML database.

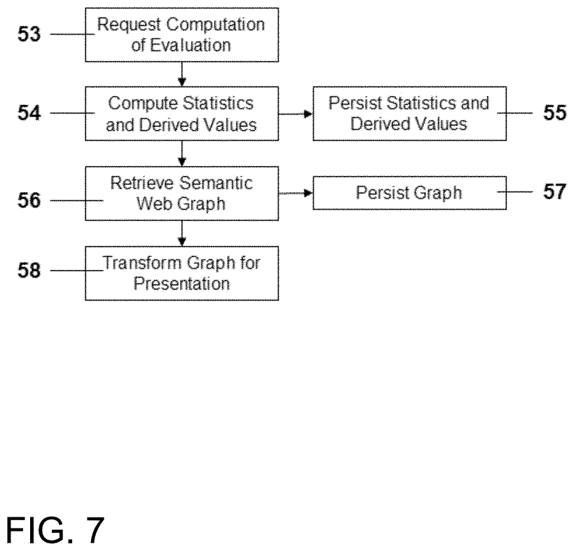

Another method, depicted in FIG. 7, manages the underlying patient and/or drug database data as semantic web triples (optionally reified and managed as quads) represented using either RDF(S) or a dialect of OWL. A process pipeline using SPARQL statements along with either XSLT or XQuery statements is invoked when a regimen evaluation is requested, retrieving an RDF(S) or OWL graph 56 which may be persisted 57 or passed and transforming 58 this to the final form of the evaluation for presentation, which may be XML, XHTML or HTML. Event handlers, developed statically, process modification events to the regimen or its evaluation/presentation and update or version the underlying Semantic Web files, triplestore or quadstore. A variation on this method employs an abstraction layer to manage the triples or quads in a relational database.

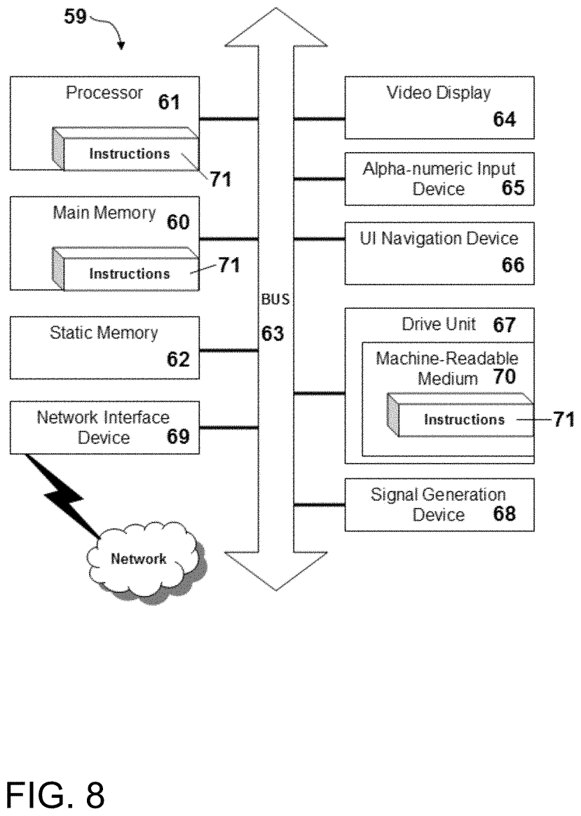

FIG. 8 illustrates a block diagram of an exemplary general purpose computing system within which a set of instructions, for causing the machine to perform any one or more of the methodologies discussed herein, may be executed. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a server computer, a client computer, a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The exemplary computing system 59 includes a processor 61 (e.g., a central processing unit (CPU) a graphics processing unit (GPU) or both), a main memory 60 and a static 62, which communicate with each other via a bus 63. The computing system 59 may further include a video display unit 64 (e.g., a liquid crystal display (LCD) or a cathode ray tube (CRT)). The computing system 59 also includes an alpha-numeric input device 65 (e.g., a keyboard), a UI navigation device 66 (e.g., a mouse), a disk drive unit 67, a signal generation device 68 (e.g., a speaker) and a network interface device 69.

The disk drive unit 67 includes a machine-readable medium 70 on which is stored one or more sets of instructions 71 (e.g., software) embodying any one or more of the methodologies, structures or functions described herein. The software may also reside, completely or at least partially, within the main memory and/or within the processor during execution thereof by the computing system, the main memory 60 and the processor 61 also constituting machine-readable media.

The software may further be transmitted or received over a network via the network interface device 69.

While the machine-readable medium 70 is shown in an exemplary embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. The term “machine-readable medium” shall accordingly be taken to include, but not be limited to, solid-state memories, optical and magnetic media, and carrier wave signals.

Table 2 provides aspects 1 through n that may be of any of the types, kinds and sorts of aspects discussed above. For example, Aspect 1 may be the attribute “Risk of Nausea (derived)” while Aspect 2 may be the attribute “Risk of Nausea (measured)” and Aspect 3 may be the attribute “Out of pocket Cost (monthly)”.

TABLE 2

| | Healthcare Regimen | | | | | |
|---|---|---|---|---|---|---|
| Healthcare | Healthcare | Healthcare | Healthcare | Healthcare | | Healthcare |
| Regimen | Regimen as | Regimen | Regimen | Regimen | | Regimen |
| Evaluation | a unit | Component 1 | Component 2 | Component 3 | . . . | Component m |
| Aspect 1 | Value¹ | Value² | Value | Value | . . . | Value |
| Aspect 2 | Value³ | Value | Value | Value | . . . | Value |
| Aspect 3 | Value⁴ | Value⁵ | Value | Value | . . . | Value |
| . . . | . . . | . . . | . . . | . . . | . . . | . . . |
| Aspect n | Value | Value | Value | Value | . . . | Value |

Value¹ is the value, presented in a chosen format, of the healthcare regimen considered as a unit with respect to the attribute “Risk of Nausea (derived)”. A value that is derived may be computed from underlying data, e.g., the risk contributions of the regimen's components; for example, this value may be “5%”. Value² is the value, presented in a chosen format, of the healthcare regimen's Component 1 with respect to the attribute “Risk of Nausea (derived).” For example, this value may be “3%”. In this example Value³ would be the value, presented in a chosen format, of the healthcare regimen considered as a unit with respect to “Risk of Nausea (measured).”

A value that is measured may be a value based on observations in the community during post market drug surveillance. For example, this value may be “8%”. Value⁴ would be the value, presented in a chosen format, of the healthcare regimen considered as a unit with respect to the attribute “Out of pocket Cost (monthly)”; for example, “$280”. Value⁵ would then be the value, presented in a chosen format, of the healthcare regimen's Component 1 with respect to the attribute “Out of pocket Cost (monthly)”; for example, “$42.”

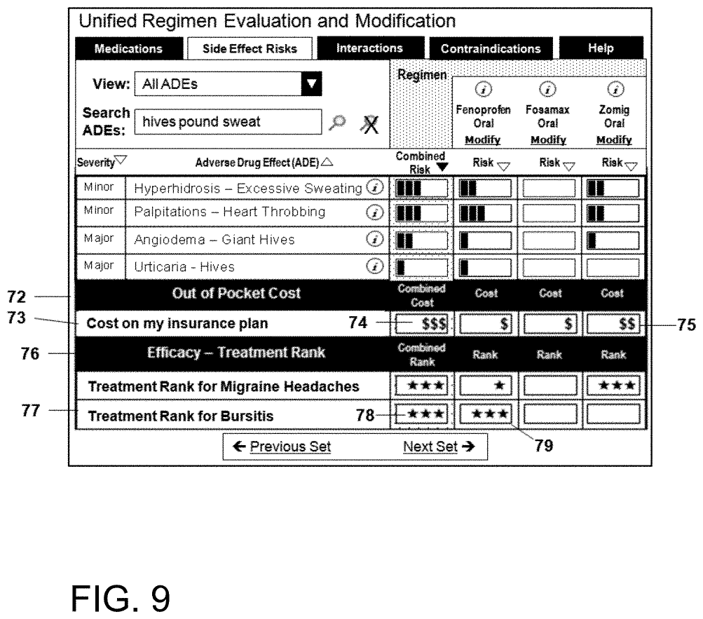

FIG. 9 illustrates a unified regimen evaluation, presentation and modification interface presenting multiple kinds of aspects of healthcare regimens, according to one embodiment. Header 72 groups together row(s) for aspects of Out of Pocket Costs. Row 73 represents the aspect of Cost on my insurance. Graph 74 depicts the value of combined cost on my insurance of the regimen as a unit (e.g., total cost across all drugs in the regimen).

Graph 75 depicts the value of the cost on my insurance of a single regimen component, in this case the drug Zomig. In this example, it may be seen at a glance that Zomig contributes the most to the cost of the regimen and that both Fenoprofen and Fosamax are comparatively less costly than Zomig and consequently contribute less cost to the regimen overall. Header 76 groups together row(s) relating to the benefit aspects of efficacy, in this case, treatment rank. Row 77 represents the aspect of treatment rank for Bursitis. Graph 78 represents that the regimen, considered as a unit, provides a three star ranking for treating Bursitis. Graph 79 represents that the individual regimen component, in this case the drug Fenoprofen, has a three star ranking for treating Bursitis. One may see the comparative merits of each regimen component, in this case drug, towards treating each of the two conditions shown.

Thus, a method and apparatus for the unified evaluation, presentation and modification of healthcare regimens have been described. Although the present invention has been described with reference to specific exemplary embodiments, it will be evident that various modifications and changes may be made to these embodiments without departing from the broader spirit and scope of the invention. Accordingly, the specification and drawings are to be regarded in an illustrative rather than a restrictive sense.

What is claimed is:

1. A method for modifying a healthcare regimen for an individual patient,
the method comprising:
determining an individual patient's healthcare regimen,
the healthcare regimen having at least three healthcare components;
storing data representing the healthcare regimen in a computing system;
computing, by a processor in the computing system, a health care regimen risk level value for the health care regimen;
identifying a modified healthcare regimen better suited to the individual patient based in part on the computed risk-level value; and
modifying the healthcare regimen of the individual patient.

2. The method of claim 1, wherein
the healthcare regimen comprises a multi-drug regimen with drug dose information;
and the step of modifying the healthcare regimen comprises
changing at least one of the dose, duration, and frequency
for at least one of the drugs of the multi-drug regimen for the individual patient.

3. The method of claim 1, wherein healthcare components are selected from the group consisting of:
prescription drugs, over-the-counter drugs, nutraceuticals, medical devices, and diagnostics.

4. The method of claim 1,
wherein the at least three healthcare components each have plurality of aspects;
and additionally comprising:
assigning, by a processor in the computing system, an evidence-based statistical first value associated with at least one of the plurality of aspects to each of the stored healthcare components;
partitioning, by a processor in the computing system, an event sample space and identifying a plurality of events associated with the first value aspect,
wherein each identified event is related to an element of a power set of all components of the healthcare regimen
and the element is composed of a subset of regimen components;
generating, by a processor in the computing system, a second value for each component associated with an identified event,
wherein the second value is a compound conditional probability of the event based on at least one of
a total probability,
a Bayesian probability value of the event, and
a Dempster-Shafer value of the event;
and wherein the step of computing a health care regimen risk level value is based on at least three values,
wherein the three values include at least one of:
a plurality of the first statistical values associated with the plurality of aspects, respectively,
the plurality of second values, and
a computation based on at least one of:
at least two statistical values associated with the aspect;
at least three of the plurality of second values;
and a combination of at least one statistical value and at least one second value; and wherein
generating, by a processor in the computing system, a regimen risk-level profile indicating a likelihood of the healthcare regimen risk associated with the healthcare regimen based on the corresponding at least one aspect and at least one event.

5. The method of claim 4, additionally comprising:
displaying on a user interface the regimen risk-level profile in a table having columns and rows, the user interface configured to adapt to a screen size by expanding and/or collapsing the rows and columns.

6. The method of claim 5, wherein
the user interface configured to adapt to a screen size by expanding and/or collapsing the rows and columns.

7. A method for automatically determining a risk impact of a healthcare aspect at the healthcare regimen level for at least three healthcare components, the method comprising:
receiving and storing in a memory a healthcare regimen having at least three or more healthcare components;
assigning, by a processor an evidence-based statistical first value associated with at least one of the plurality of aspects to each of the stored healthcare components;
partitioning an event sample space and identifying a plurality of events associated with the first value aspect, wherein each identified event is related to an element of a power set of all components of the healthcare regimen and the element is composed of a subset of regimen components;
generating a second value for each component associated with an identified event, wherein the second value is a compound conditional probability of the event based on at least one of
a total probability,
a Bayesian probability value of the event, and
a Dempster-Shafer value of the event;
computing a health care regimen risk level value based on at least three values, wherein the three values include at least one of:
a plurality of the first statistical values associated with the plurality of aspects, respectively,
the plurality of second values, and
a computation based on at least one of:
at least two statistical values associated with the aspect;
at least three of the plurality of second values;
and a combination of at least one statistical value and
at least one second value;
dynamically generating a regimen risk-level profile indicating a likelihood of the healthcare regimen risk associated with the healthcare regimen based on the corresponding at least one aspect and at least one event; and
displaying on a user interface the regimen risk-level profile in a table having columns and rows, the user interface configured to adapt to a screen size by expanding and/or collapsing the rows and columns.

8. The method of claim 7, wherein
the step of computing a health care regimen risk level value is performed using a combinatorial component method.

9. The method of claim 7, wherein
the step of computing a health care regimen risk level value is performed using an alternate cross-check method.

10. The method of claim 7, wherein
the step of computing a health care regimen risk level value is performed in a single step.

11. The method of claim 7, wherein
the step of computing a health care regimen risk level value is performed in an iteration of multiple steps.

12. The method of claim 7, wherein
the step of computing a health care regimen risk level value employs one or more computing methods selected from the group consisting of
a component method, an alternate method, single step,
iteration, sequential processing, and parallel processing.

13. A computer system for the evaluation, presentation and modification of a drug regimen, the system comprising:
a drive unit configured to store drug information;
a processor configured to execute a computer program configured to determine a plurality of adverse drug effects corresponding to the plurality of drugs based on the drug information; and
a video display configured to display a graphical user interface of the computer program,
the graphical user interface configured
to display a table having cells displayed in columns and rows,
each row corresponding to a respective one of the plurality of adverse drug effects and each column corresponding to one of a plurality of drugs in a drug regimen,
each cell displaying a risk level of the respective one of the plurality of adverse drug effects for the respective one of the plurality of drugs,
the video display further configured to, in response to user input,
allow for expanding and collapsing one or more of the columns.

14. The computer system of claim 13, wherein
the graphical user interface is configured to expand, upon user input,
columns displaying the risk level of the respective one of the plurality of adverse drug effects for each of the plurality of drugs
to include columns displaying the risk level of the respective one of the plurality of adverse drug effects for one or more ingredients for each of the plurality of drugs.

15. The computer system of claim 14, wherein
the graphical user interface is configured to collapse, upon user input,
the columns displaying the risk level of the respective one of the plurality of adverse drug effects for one or more ingredients for each of the plurality of drugs.

16. The computer system of claim 13, wherein
the graphical user interface is configured to expand, upon user input, a row corresponding to one of the plurality of adverse drug effects
to reveal a plurality of additional rows,
each of the plurality of additional rows corresponding to sub-types of adverse drug effects that are each types of the one of the plurality of adverse drug effects.

17. The computer system of claim 16, wherein
the graphical user interface is further configured to collapse, upon user input, the plurality of additional rows such that only the one of the plurality of adverse drug effects is visible.

18. The computer system of claim 13, wherein
the graphical user interface is configured to expand the rows and columns to reveal a plurality of row levels and column levels, respectively.

19. The computer system of claim 13, wherein
the cells display statistical variance across the plurality of drugs.

20. The computer system of claim 19, wherein
the statistical variance comprises one or more selected from the group of mean, median, standard deviation, minimum and maximum.
